(19) United States
(12) Patent Application Publication
(10) Pub. No.: US 2026/0207048 A1
(43) Pub. Date: Jul. 23, 2026

(54) OPTICAL BIOMETER AND OPERATING METHOD THEREOF

(71) Applicant: CRYSTALVUE MEDICAL CORPORATION, Taoyuan City (TW)

(72) Inventors: YEN-JEN CHANG, Taoyuan City (TW); SUNG-YANG WEI, Taoyuan City (TW); CHUN-NAN LIN, Taoyuan City (TW); CHUNG-PING CHUANG, Taoyuan City (TW); WILLIAM WANG, Taoyuan City (TW); TUNG-YU LEE, Taoyuan City (TW)

(21) Appl. No.: 19/452,601

(22) Filed: Jan. 19, 2026

Related U.S. Application Data
(60) Provisional application No. 63/748,750, filed on Jan. 23, 2025.

(51) Int. Cl. A61B 3/10; A61B 3/14

(52) U.S. Cl. CPC A61B 3/1005; A61B 3/14

(57) ABSTRACT

An optical biometer is disclosed. In the optical biometer, an optical coupling module receives an incident light from a light source module and emits a reference light and a sensing light. The reference arm changes a position of an optical path switching unit in a first axis and a second axis through the drive of first and second motors to reflect the reference light to generate a first reflected light. The sensing arm transmits the sensing light to an eye and transmits a second reflected light from the eye. The optical coupling module interferes the first and second reflected lights to generate an optical interference signal. The detection module generates a detection result related to the eye according to the optical interference signal. The reference arm selectively generates a first or second real time position signal corresponding to a first or second interface of eye through a switching mechanism.

### CROSS REFERENCE TO RELATED APPLICATION

This application claims priority to and the benefit of U.S. Application Serial No. 63/748,750, having a filing date of January 23, 2025, the entire content of which is incorporated herein by reference as if fully set forth below in its entirety and for all applicable purposes.

### TECHNICAL FIELD

The invention relates to a biometer, more particularly, to an optical biometer and an operating method thereof.

### BACKGROUND

In general, when a conventional optical coherence tomography (OCT) biometer is used to measure axial length, it is necessary to collect real-time position signals of the corneal surface and fundus firstly and calculate a distance between them. As shown in FIG. 1, in the conventional design of a reference arm RA, a single motor MT is used to drive optical components to move throughout their full stroke, thereby sequentially collecting eye feature position signals required for calculating the axial length, such as a first real-time position signal CN corresponding to the cornea and a second real-time position signal RT corresponding to the retina.

However, because the linear speed at which the single screw SC, controlled by the single motor MT, linearly transforms the position of the optical components along a single axis AX is limited, it is often difficult to prevent eye movement from affecting the collected first and second real-time position signals CN and RT during the entire measurement process, thus leading to errors in the calculation of the axial length. While this calculation error can be overcome by increasing the number of measurements and the duration of the measurement, it also significantly increases the measurement time and exacerbates the discomfort in the eyes of the test subjects and it is therefore desirable to improve this situation.

### SUMMARY

In view of this, an optical biometer and an operating method thereof are proposed in the invention to effectively solve the above-mentioned problems in the prior art.

An embodiment of the invention is an optical biometer. In this embodiment, the optical biometer includes a light source module, an optical coupling module, a reference arm, a sensing arm and a detection module. The light source module is configured to emit an incident light. The optical coupling module is disposed corresponding to the light source module and configured to receive the incident light and emit a reference light and a sensing light respectively. The reference arm is disposed corresponding to the optical coupling module. The reference arm includes an optical path switching unit. The reference arm is configured to change a position of the optical path switching unit along a first axis and a second axis through a drive of a first motor and a second motor respectively to reflect the reference light to generate a first reflected light to the optical coupling module. The sensing arm is disposed corresponding to the optical coupling module and configured to transmit the sensing light to an eye under test and transmit a second reflected light generated by the eye under test reflecting the sensing light to the optical coupling module. The optical coupling module interferes the first reflected light and the second reflected light to generate an optical interference signal. The detection module is disposed corresponding to the optical coupling module and configured to generate a detection result related to the eye under test according to the optical interference signal.

In an embodiment, a first interface of the eye under test is a cornea and the second interface of the eye under test is a retina; an axial length of the eye under test is a distance between the retina and the cornea, and the axial length is calculated based on a first real-time position signal corresponding to the first interface and a second real-time position signal corresponding to the second interface.

In an embodiment, the optical path switching unit switches between a first optical path and a second optical path, such that the reference arm collects the first real-time position signal and the second real-time position signal through the first optical path and the second optical path respectively.

In an embodiment, the optical path switching unit performs optical path switching by using a design of reflecting mirrors, cubes, mirrors moving in and out or rotating mechanisms.

In an embodiment, the sensing arm includes a first beam splitter, a second beam splitter, a first mirror and a second mirror, and uses a design of the first beam splitter, the second beam splitter, the first mirror and the second mirror to allow the sensing light to be emitted to the first interface and the second interface of the eye under test to generate an optical path difference.

Another embodiment of the invention is a method for operating an optical biometer. In this embodiment, the optical biometer includes a light source module, an optical coupling module, a reference arm, a sensing arm and a detection module. The reference arm includes an optical path switching unit, the method includes steps of: the light source module emitting an incident light; the optical coupling module disposed corresponding to the light source module receiving the incident light and emitting a reference light and a sensing light respectively; the reference arm disposed corresponding to the optical coupling module changing a position of the optical path switching unit along a first axis and a second axis through a drive of a first motor and a second motor respectively to reflect the reference light to generate a first reflected light to the optical coupling module; the sensing arm disposed corresponding to the optical coupling module transmitting the sensing light to an eye under test and the sensing arm also transmitting a second reflected light generated by the eye under test reflecting the sensing light to the optical coupling module; the optical coupling module interfering the first reflected light and the second reflected light to generate an optical interference signal; and the detection module disposed corresponding to the optical coupling module generating a detection result related to the eye under test according to the optical interference signal.

Compared to the prior art, the optical biometer and its operating method proposed in this invention can achieve linear transformation of the position of the optical path switching element in a dual-axis manner by the transmission of different motors through the optical path switching mechanism of the reference arm, thereby collect real-time position signals of different interfaces of the subject's eye separately. This avoids the disadvantage of conventional reference arm that collect real-time position signals of different interfaces of the subject's eye simultaneously in a single axis using a single motor. Therefore, it can effectively eliminate the error in calculating the axial length caused by the unavoidable movement of the eyeball, and can also significantly shorten the overall measurement time and reduce the discomfort of the subject's eyes.

The advantages and spirit of the present invention can be further understood from the following detailed description of the invention and the accompanying drawings.

### BRIEF DESCRIPTION OF THE DRAWINGS

The accompanying drawings of this invention are described below:

FIG. 1 is a schematic diagram of a reference arm system driven by a single motor in the prior art.

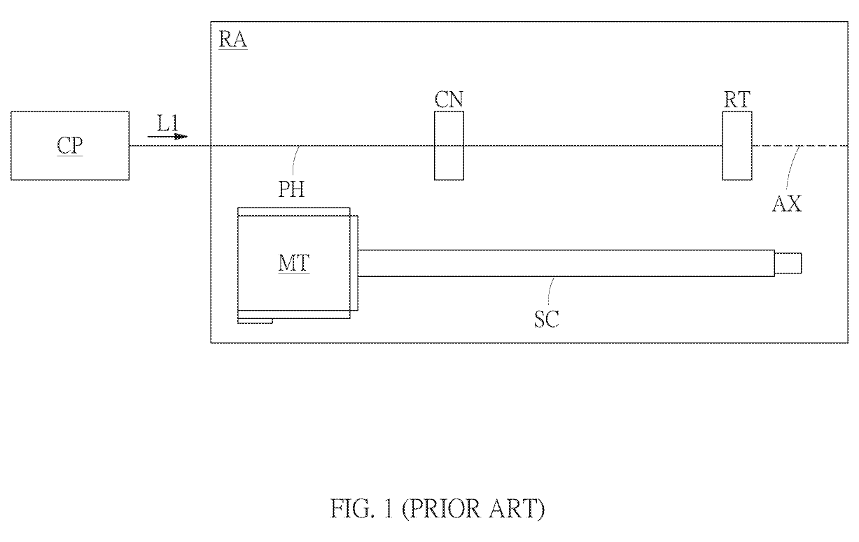

FIG. 2 is a schematic diagram of an optical biometer in an embodiment of the invention.

FIG. 3 is a schematic diagram of the reference arm of an optical biometer in an embodiment of the invention.

FIG. 4 is a flowchart of the optical biometer operating method in an embodiment of the invention.

### DETAILED DESCRIPTION

Reference will now be made in detail to exemplary embodiments of the invention, examples of which are illustrated in the accompanying drawings. Elements/components using the same or similar reference numerals in the drawings and embodiments are used to represent the same or similar parts.

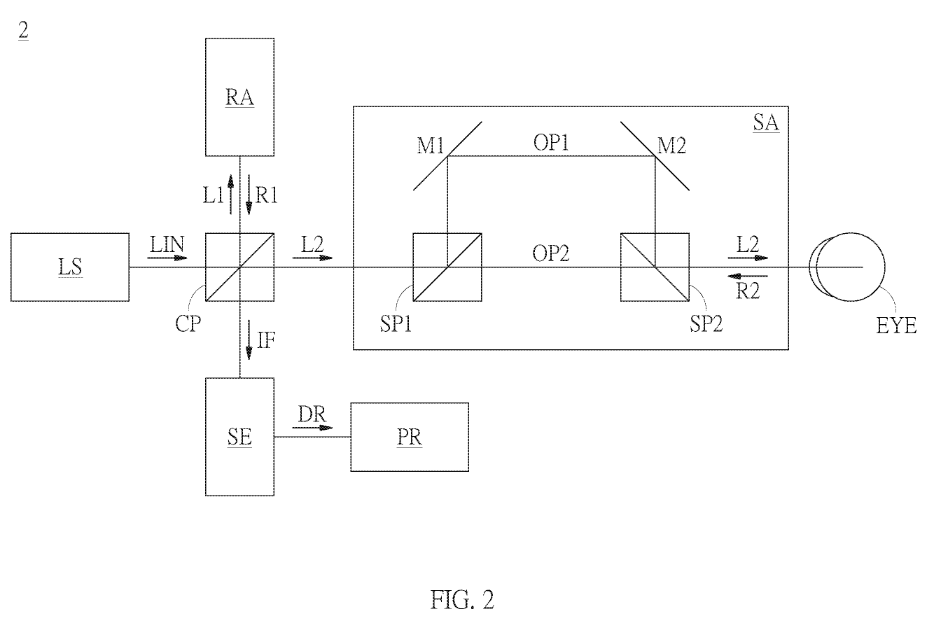

A specific embodiment of the invention is an optical biometer. In this embodiment, the optical biometer can be an optical coherence tomography (OCT) biometer, but not limited to this. Please refer to FIG. 2, which illustrates a schematic diagram of the optical biometer in this embodiment.

As shown in FIG. 2, the optical biometer 2 includes a light source module LS, an optical coupling module CP, a reference arm RA, a sensing arm SA, a detection module SE and a processing module PR. The optical coupling module CP is disposed between the light source module LS and the sensing arm SA, and the optical coupling module CP is also disposed between the reference arm RA and the detection module SE. The sensing arm SA is disposed between the optical coupling module CP and an eye EYE under test. The detection module SE is coupled to the processing module PR. In practical applications, there are no specific restrictions on the type of light source in the light source module LS; the optical coupling module CP can include a beam splitter, but not limited to this; the processing module PR can be a microcontroller unit (MCU) or a central processing unit (CPU), but not limited to this.

The light source module LS is configured to emit an incident light LIN to the optical coupling module CP. When the incident light LIN reaches the optical coupling module CP, the optical coupling module CP is configured to split the incident light LIN into a reference light L1 and a sensing light L2, and then emit the reference light L1 toward the reference arm RA and emit the sensing light L2 toward the sensing arm SA respectively. The reference arm RA is configured to reflect the reference light L1 to generate a first reflected light R1 to the optical coupling module CP. The sensing arm SA is configured to transmit the sensing light L2 to the eye EYE under test, and the sensing arm SA also transmits a second reflected light R2 generated by the eye EYE under test reflecting the sensing light L2 to the optical coupling module CP.

In this embodiment, the sensing arm SA includes a first beam splitter SP1, a second beam splitter SP2, a first mirror M1 and a second mirror M2. The first beam splitter SP1 is disposed between the optical coupling module CP and the second beam splitter SP2. The second beam splitter SP2 is positioned between the first beam splitter SP1 and the eye EYE under test. The first mirror M1 is disposed relative to the first beam splitter SP1 and the second mirror M2. The second mirror M2 is disposed relative to the first mirror M1 and the second beam splitter SP2. The design of the first beam splitter SP1, the second beam splitter SP2, the first mirror M1 and the second mirror M2 allows the sensing light L2 to be emitted towards different interfaces of the eye EYE under test, thereby an optical path difference is generated.

For example, the design of the first beam splitter SP1, the second beam splitter SP2, the first mirror M1 and the second mirror M2 can form two paths OP1 and OP2. During the transmission of the sensing light L2 from the sensing arm SA to the eye EYE under test, if the sensing light L2 is transmitted along the path OP1, the sensing light L2 will sequentially pass through the first beam splitter SP1, the first mirror M1, the second mirror M2 and the second beam splitter SP2 before reaching the eye EYE under test. If the sensing light L2 is transmitted along the path OP2, the sensing light L2 will sequentially pass through the first beam splitter SP1 and the second beam splitter SP2 before reaching the eye EYE under test.

In practical applications, the sensing arm SA can also switch between the paths OP1 and OP2 via a switching mechanism, allowing the sensing light L2 to selectively travel along the path OP1 or the path OP2 towards the first interface (e.g., the cornea) or the second interface (e.g., the retina) of the eye EYE under test, thereby creating an optical path difference between the second reflected light R2 reflected from the first interface (e.g., the cornea) and the second interface (e.g., the retina) of the eye EYE under test.

Similarly, during the process of the sensing arm SA transmitting the second reflected light R2 reflected from the first interface (e.g., the cornea) or second interface (e.g., the retina) of the eye EYE under test to the optical coupling module CP, the second reflected light R2 can also be selectively transmitted to the optical coupling module CP along the path OP1 or the path OP2. If the second reflected light R2 is transmitted along the path OP1, it will sequentially pass through the second beam splitter SP2, the second mirror M2, the first mirror M1 and the first beam splitter SP1 before being transmitted to the optical coupling module CP; if the second reflected light R2 is transmitted along path OP2, it will sequentially pass through the second beam splitter SP2 and the first beam splitter SP1 before reaching the optical coupling module CP.

In practical applications, the sensing arm SA can also switch between the paths OP1 and OP2 via a switching mechanism, allowing the second reflected light R2 reflected from either the first interface (e.g., the cornea) or the second interface (e.g., the retina) of the eye EYE under test to be selectively transmitted along the path OP1 or the path OP2 to the optical coupling module CP.

When the optical coupling module CP receives the first reflected light R1 and the second reflected light R2, the optical coupling module CP is configured to interfere the first reflected light R1 and the second reflected light R2 to generate an optical interference signal IF, and then the optical coupling module CP transmits the optical interference signal IF to the detection module SE. The detection module SE is then configured to generate a detection result DR for the eye EYE under test based on the optical interference signal IF, and then the detection module SE transmits the detection result DR to the processing module PR. The processing module PR is configured to analyze and process the detection result DR and obtain a first real-time position signal corresponding to the first interface (e.g., the cornea) of the eye EYE under test and a second real-time position signal corresponding to the second interface (e.g., the retina) of the eye EYE under test. The distance between the first interface (e.g., the cornea) and the second interface (e.g., the retina) of the eye EYE under test can be calculated based on the first and second real-time position signals, i.e., the axial length of the eye EYE under test, but not limited to this.

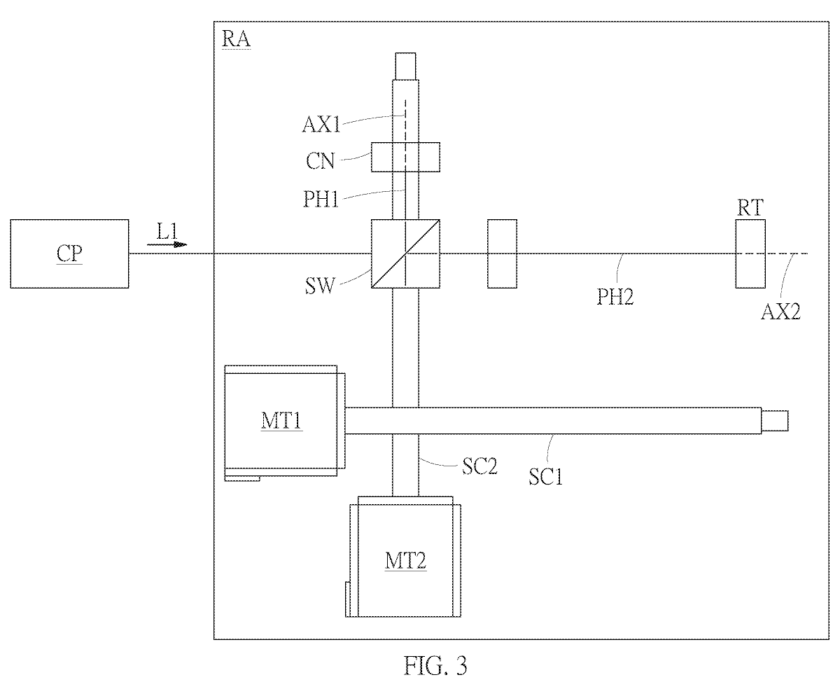

Please refer to FIG. 3, which illustrates a schematic diagram of the reference arm of the optical biometer in an embodiment of the invention. As shown in FIG. 3, the reference arm RA controls the first screw SC1 and the second screw SC2 to linearly change the position of the optical path switching element along the first axis AX1 and the second axis AX2 through the transmission of the first motor MT1 and the second motor MT2 respectively to collect the first real-time position signal CN corresponding to the first interface (e.g., the cornea) of the eye EYE under test and the second real-time position signal RT corresponding to the second interface (e.g., the retina) of the eye EYE under test.

In this embodiment, the reference arm RA can include a switching mechanism. For example, the reference arm RA can include an optical path switching unit SW, which switches between a first optical path PH1 and a second optical path PH2. When the optical path switching unit SW switches to the first optical path PH1, the reference light L1 emitted from the optical coupling module CP into the reference arm RA is transmitted along the first optical path PH1 to collect the first real-time position signal CN corresponding to the first interface (e.g., the cornea) of the eye EYE under test. When the optical path switching unit SW switches to the second optical path PH2, the reference light L1 emitted from the optical coupling module CP into the reference arm RA is transmitted along the second optical path PH2 to collect the second real-time position signal RT corresponding to the second interface (e.g., the retina) of the eye EYE under test, but not limited to this.

In practical applications, the optical path switching unit SW of the reference arm RA can be designed in various ways, such as a mirror, a cube, a mirror moving in and out, or a rotating mechanism, to switch the optical path, but not limited to this. Furthermore, the first axis AX1 and the second axis AX2 can be parallel to the first optical path PH1 and the second optical path PH2 respectively, but not limited to this.

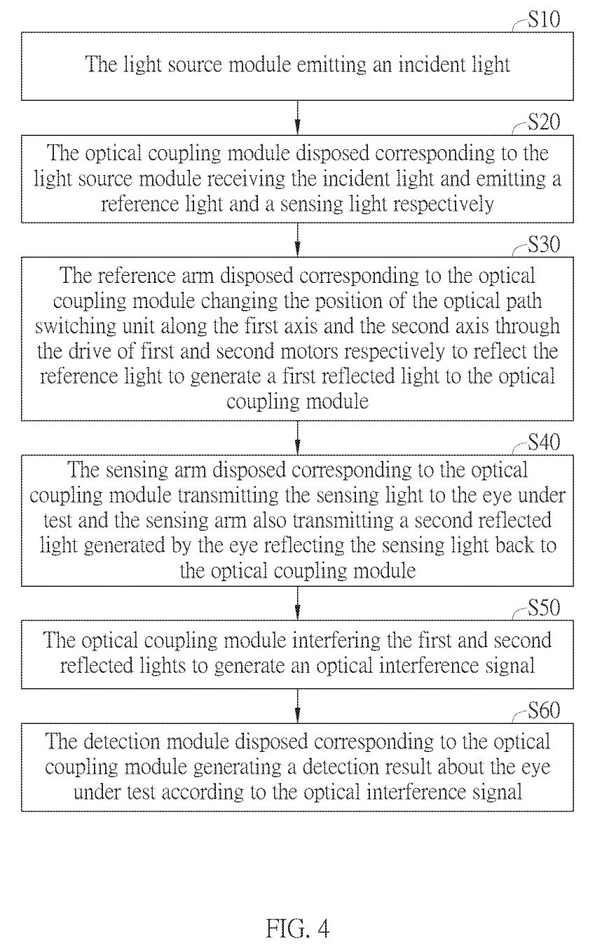

Another specific embodiment of the invention is a method for operating an optical biometer. In this embodiment, the optical biometer can be an optical coherence interferometry biometer, but not limited to this. The optical biometer includes a light source module, an optical coupling module, a reference arm, a sensing arm and a detection module. The reference arm includes an optical path switching unit. Please refer to FIG. 4, which illustrates a flowchart of the optical biometer operating method in this embodiment.

As shown in FIG. 4, the method in this embodiment includes the following steps:

Step S10: the light source module emitting an incident light;

Step S20: the optical coupling module disposed corresponding to the light source module receiving the incident light and emitting a reference light and a sensing light respectively;

Step S30: the reference arm disposed corresponding to the optical coupling module changing the position of the optical path switching unit along the first axis and the second axis through the drive of first and second motors respectively to reflect the reference light to generate a first reflected light to the optical coupling module;

Step S40: the sensing arm disposed corresponding to the optical coupling module transmitting the sensing light to the eye under test and the sensing arm also transmitting a second reflected light generated by the eye reflecting the sensing light back to the optical coupling module;

Step S50: the optical coupling module interfering the first and second reflected lights to generate an optical interference signal; and

Step S60: the detection module disposed corresponding to the optical coupling module generating a detection result about the eye under test according to the optical interference signal.

In practical applications, the sensing arm can include a first beam splitter, a second beam splitter, a first mirror and a second mirror. The design of the first beam splitter, the second beam splitter, the first mirror and the second mirror allows the sensing light to be emitted towards the first and second interfaces of the eye under test, thereby generating an optical path difference, but not limited to this.

It should be noted that the reference arm can control the first and second screws to linearly change the position of the optical path switching unit along the first axis and the second axis respectively through the transmission of the first and second motors, so as to collect a first real-time position signal corresponding to the first interface (e.g., the cornea) and a second real-time position signal corresponding to the second interface (e.g., the retina) of the eye under test. Since the axial length of the eye under test is the distance between the cornea and the retina, the axial length can be calculated based on the aforementioned first and second real-time position signals, but not limited to this. Furthermore, the first axis and the second axis can be parallel to the first and second optical paths respectively, but not limited to this.

In an embodiment, the reference arm can selectively generate the first real-time position signal corresponding to the first interface of the eye under test or the second real-time position signal corresponding to the second interface of the eye under test through a switching mechanism. For example, the reference arm can switch between a first optical path and a second optical path via an optical path switching unit, such that the reference arm collects the first real-time position signal and the second real-time position signal through the first optical path and the second optical path respectively, but not limited to this.

In other embodiments, the optical path switching unit can be designed in various ways, such as a reflector, a cube, a mirror moving in and out, or a rotating mechanism, to perform optical path switching, but not limited to this.

Compared to the prior art, the optical biometer and its operating method proposed in this invention can achieve linear transformation of the position of the optical path switching element in a dual-axis manner by the transmission of different motors through the optical path switching mechanism of the reference arm, thereby collect real-time position signals of different interfaces of the subject's eye separately. This avoids the disadvantage of conventional reference arm that collect real-time position signals of different interfaces of the subject's eye simultaneously in a single axis using a single motor. Therefore, it can effectively eliminate the error in calculating the axial length caused by the unavoidable movement of the eyeball, and can also significantly shorten the overall measurement time and reduce the discomfort of the subject's eyes.

The above-described embodiments are only for the convenience of illustrating the present invention and are not intended to limit it. Various simple modifications and alterations made by those skilled in the art based on the claims and description of the present invention without departing from the spirit and scope of the present invention should still be included in the following claims.

What is claimed is:

1. An optical biometer, comprising:
a light source module, configured to emit an incident light;
an optical coupling module, disposed corresponding to the light source module and configured to receive the incident light and emit a reference light and a sensing light respectively;
a reference arm, disposed corresponding to the optical coupling module, the reference arm comprising an optical path switching unit, the reference arm being configured to change a position of the optical path switching unit along a first axis and a second axis through a drive of a first motor and a second motor respectively to reflect the reference light to generate a first reflected light to the optical coupling module;
a sensing arm, disposed corresponding to the optical coupling module and configured to transmit the sensing light to an eye under test and transmit a second reflected light generated by the eye under test reflecting the sensing light to the optical coupling module, the optical coupling module interfering the first reflected light and the second reflected light to generate an optical interference signal; and
a detection module, disposed corresponding to the optical coupling module and configured to generate a detection result related to the eye under test according to the optical interference signal.

2. The optical biometer according to claim 1, wherein a first interface of the eye under test is a cornea and the second interface of the eye under test is a retina; an axial length of the eye under test is a distance between the retina and the cornea, and the axial length is calculated based on a first real-time position signal corresponding to the first interface and a second real-time position signal corresponding to the second interface.

3. The optical biometer according to claim 2, wherein the optical path switching unit switches between a first optical path and a second optical path, such that the reference arm collects the first real-time position signal and the second real-time position signal through the first optical path and the second optical path respectively.

4. The optical biometer according to claim 1, wherein the optical path switching unit performs optical path switching by using a design of reflecting mirrors, cubes, mirrors moving in and out or rotating mechanisms.

5. The optical biometer according to claim 1, wherein the sensing arm comprises a first beam splitter, a second beam splitter, a first mirror and a second mirror, and uses a design of the first beam splitter, the second beam splitter, the first mirror and the second mirror to allow the sensing light to be emitted to the first interface and the second interface of the eye under test to generate an optical path difference.

6. A method for operating an optical biometer, the optical biometer comprising a light source module, an optical coupling module, a reference arm, a sensing arm and a detection module, the reference arm comprising an optical path switching unit, the method comprising steps of:
the light source module emitting an incident light;
the optical coupling module disposed corresponding to the light source module receiving the incident light and emitting a reference light and a sensing light respectively;
the reference arm disposed corresponding to the optical coupling module changing a position of the optical path switching unit along a first axis and a second axis through a drive of a first motor and a second motor respectively to reflect the reference light to generate a first reflected light to the optical coupling module;
the sensing arm disposed corresponding to the optical coupling module transmitting the sensing light to an eye under test and the sensing arm also transmitting a second reflected light generated by the eye under test reflecting the sensing light to the optical coupling module;
the optical coupling module interfering the first reflected light and the second reflected light to generate an optical interference signal; and
the detection module disposed corresponding to the optical coupling module generating a detection result related to the eye under test according to the optical interference signal.

7. The method according to claim 6, wherein a first interface of the eye under test is a cornea and the second interface of the eye under test is a retina; an axial length of the eye under test is a distance between the retina and the cornea, and the axial length is calculated based on a first real-time position signal corresponding to the first interface and a second real-time position signal corresponding to the second interface.

8. The method according to claim 7, wherein the optical path switching unit switches between a first optical path and a second optical path, such that the reference arm collects the first real-time position signal and the second real-time position signal through the first optical path and the second optical path respectively.

9. The method according to claim 6, wherein the optical path switching unit performs optical path switching by using a design of reflecting mirrors, cubes, mirrors moving in and out or rotating mechanisms.

10. The method according to claim 6, wherein the sensing arm comprises a first beam splitter, a second beam splitter, a first mirror and a second mirror, and uses a design of the first beam splitter, the second beam splitter, the first mirror and the second mirror to allow the sensing light to be emitted to the first interface and the second interface of the eye under test to generate an optical path difference.
